(19) United States
(12) Patent Application Publication
(10) Pub. No.: US 2004/0082900 A1
(43) Pub. Date: Apr. 29, 2004

(54) Proportional control device for a hydrocephalus shunt

(76) Inventor: Edward H. Luttich, Goleta, CA (US)

Correspondence Address: KENNETH J. HOVET, NORDMAN, CORMANY, HAIR & COMPTON, P.O. BOX 9100, 1000 TOWN CENTER DRIVE, OXNARD, CA, 93031-9100, US

(21) Appl. No.: 10/373,231

(22) Filed: Feb. 24, 2003

Related U.S. Application Data
(60) Provisional application No. 60/359,449, filed on Feb. 23, 2002.

(51) Int. Cl. A61M 5/00

(52) U.S. Cl. 604/9

(57) ABSTRACT

A cerebral spinal fluid (CSF) shunt is provided, comprising a ventricular cavity for receiving CSF in a drainage chamber for passing the CSF to a patient's abdominal cavity. An anti-syphon valve controls CSF flow from the ventricular cavity to the drainage chamber. The valve utilizes a thin, flexible portion of the ventricular cavity which operates an underlying pressure control membrane. The membrane overlies a valve opening that permits CSF flow from the ventricular cavity into the drainage chamber. In place of the membrane, a spring-biased ball can be used to open and close the valve opening.

This application claims priority from Provisional Application No. 60/359,449, filed Feb. 23, 2002.

### BACKGROUND OF THE INVENTION

1. Field of the Invention

The present invention pertains generally to medical devices. More particularly, the invention concerns hydrocephalus shunts for maintaining normal cerebrospinal fluid (CSF) pressure in the brain. The present invention is particularly, but not exclusively, useful as a hydrocephalus shunt that limits an undesirable siphoning phenomena that is common in implanted hydrocephalus shunts.

2. Description of Related Art

To treat the condition of excessive fluid pressure in the human brain (hydrocephalus) a system referred to as a shunt is generally implanted in the body of the patient. A hydrocephalus shunt typically consists of two catheter tubes that are interconnected by a pressure control valve. The first catheter, referred to as the ventricular catheter, has one end inserted through a hole that is drilled in the skull and into a ventricle of the brain. The other end of the ventricular catheter is connected to the pressure control valve, which is implanted under the scalp. The second catheter, known as the drainage catheter, has one end connected to the pressure control valve, and the other end of the drainage catheter empties into a lower body cavity, usually the peritoneal cavity.

The pressure control valve is designed to open at a predetermined pressure to allow drainage of cerebrospinal fluid (CSF) from the ventricle of the brain to the peritoneal cavity, where it is re-absorbed by the body. This maintains the CSF pressure in the brain within a normal range of values. The pressure control valve can be of a number of different designs. Some pressure control valves use flexible elastomeric membranes to flex open under pressure, while others use ball and spring designs or other means to open in order to control CSF pressure. To reduce risk of infection, the valves are designed to allow CSF flow only out of the brain, and not back into the brain.

An additional effect occurs when CSF flows through the shunt, not because of a positive pressure in the brain, but due to the negative (suction) pressure created because the lower end of the drainage catheter is at a lower level then the end of the ventricular catheter in the brain. This effect is commonly known as the siphoning effect and causes CSF to drain even when the CSF pressure in the brain is within normal parameters. The siphoning effect varies with the position of the patient, being the most extreme siphoning effect occurs when the patient is upright, while there is virtually no siphon effect when the patient is lying down. In all situations, however, the siphoning effect is extremely undesirable and must be eliminated or kept to an absolute minimum.

To control unwanted siphoning, shunt systems are often equipped with a second valve (a siphon control valve) placed in line with the pressure control valve in order to prevent or reduce siphoning. The basic principle is generally one or more flexible walls of a chamber surrounding a port through which the fluid must flow to drain. The flexible walls are designed to collapse onto and occlude the port when negative pressure is experienced. This provides additional flow resistance to counteract the effect of the negative pressure, while still allowing drainage under positive pressure from the brain.

The type of siphon control device described above, however, has a natural tendency to operate in an on-off fashion, since it covers and uncovers an open port during operation. This leads to different response of the shunt system depending on the position of the patient.

There is, therefore, a need for a cerebrospinal fluid shunt that is responsive to the cerebrospinal fluid pressure in a patient while avoiding the siphoning effect that occurs when a drain is at a lower level than the rest of the shunt. There is also a need for a cerebrospinal fluid shunt that operates in a gradual or analog fashion rather than an on/off fashion. Additionally, the need exists for a cerebrospinal fluid shunt that exhibits a greater cooperation between responses to cerebrospinal fluid pressure and resistance to siphoning.

### SUMMARY OF THE INVENTION

The present invention is directed to a cerebrospinal fluid shunt that is responsive to the cerebrospinal fluid pressure in a patient while avoiding the siphoning effect that occurs when a drain is at a lower level than the shunt. The invention is also directed to a cerebrospinal fluid shunt that operates in a graduated or analog fashion rather than an on/off fashion, and exhibits a greater cooperation between responses to cerebrospinal fluid pressure and resistance to siphoning. Still further, the invention provides for a siphon control member that can be oriented so that it moves parallel to the main tissue plane, such as a patient's scalp. This is an advantageous feature not found in the prior art, because it significantly reduces interference with movement of the siphon control member from surrounding tissue.

The proposed invention is a valve having both the pressure control and siphon control members in the same valve chamber, such that the siphon control member acts to reinforce the resistance of the pressure control member instead of acting upon a separate port in the flow path. The pressure control valve of the present invention can have different characteristic designs for the different valve types. Embodiments illustrated herein include resilient membrane valves and ball and spring valves. Those skilled in the art will recognize that the principles illustrated herein can be applied to other valve types as well.

A pressure control valve for maintaining a proper cerebrospinal fluid pressure in the brain comprises a ventricular cavity for storing cerebrospinal fluid; a drainage chamber for receiving cerebrospinal fluid from the ventricular cavity; a port (orifice) connecting the ventricular cavity to the drainage chamber such that the ventricular cavity and the drainage chamber are in fluid communion when the port is open; a first pressure responsive member configured to urge an opening of the port in an amount proportional to a first pressure differential, the first pressure differential being measured from a pressure in the ventricular cavity to a pressure in the drainage chamber; and a second pressure responsive member configured to resist an opening of the port in an amount proportional to a second pressure differential, the second pressure differential being measured from an ambient body pressure proximate the pressure control valve to the pressure in the drainage chamber. For design purposes, it will be appreciated that the surface area of the second pressure responsive member should be at least equal to, and preferably more than, the open area defined by the port.

According to one embodiment, the first pressure responsive member comprises a pressure control membrane covering the port, and the second pressure responsive member is a siphon control membrane configured to impart a resistive force on the pressure control membrane, the resistive force being proportional to the second pressure differential. A dissimilar material is advantageously disposed between the siphon control membrane and the pressure control membrane.

In another embodiment, the first pressure responsive member comprises a sealing ball configured to regulate a cross-sectional area through which the cerebrospinal fluid can flow through the port from the ventricular cavity to the drainage chamber according to an aggregation of forces acting on the sealing ball against the port. A first force is formed from the first pressure differential.

The pressure control valve further comprises a compressive member disposed between the sealing ball and the second pressure responsive member. The compressive member is thereby configured to impart a second force directed to press the sealing ball into the port to reduce the cross-sectional area. When the aggregation of forces is less than or equal to zero, the ball will seat against the port such that no cerebrospinal fluid can pass through the port. When the aggregation of forces exceeds a preset value, the ball will unseat from the port, thereby allowing a fluid flow through the port from the ventricular cavity to the drainage chamber. The compressive member may comprise a helical spring or a substantially rigid member.

A biasing member is advantageously configured to control a displacement offset between the compressive member and the second pressure responsive member. The biasing member may comprise a material selected from a group consisting of electrostrictive materials and magnetostrictive materials. The member may also comprise a linear actuator or an oscillating device used to rotate a cam.

A method of controlling a proper cerebrospinal fluid pressure in the brain of a subject comprises the steps of channeling cerebrospinal fluid from a brain to a ventricular cavity; generating a first fluid pressure within the ventricular cavity; exerting a first force on a fluid control member according to a first pressure differential between a fluid pressure in the ventricular cavity and a fluid pressure within a drainage chamber; exerting a second force on the fluid control member according to a second pressure differential; and controlling a rate of fluid flow from the ventricular cavity to the drainage chamber through an orifice.

The rate of fluid flow may be proportional to a first force, and inversely proportional to a second force. The second pressure differential is related to a difference in an ambient pressure in the brain of a patient and the fluid pressure within the drainage chamber.

The fluid control member can be a membrane configured to controllably seal the orifice, or a sealing ball configured to controllably seal the orifice. The method advantageously comprises the steps of generating a control signal to adjust an adjustable offset bias and biasing the second force through the adjustable offset bias. The cerebrospinal fluid may be drained from the drainage chamber into a peritoneal cavity.

A pressure control apparatus for maintaining a desired cerebrospinal fluid pressure within the brain of a patient, the apparatus comprising a catheter for channeling cerebrospinal fluid from the brain to a ventricular cavity; a drainage chamber; an orifice for channeling cerebrospinal fluid from the ventricular cavity to the drainage chamber; and a fluid flow control member configured to regulate a size of an opening through the orifice according to an aggregate of a first and second force acting on the fluid flow control member, wherein a first force is related to a first pressure differential, and a second force is related to a second pressure differential.

The first pressure differential is derived from a difference between a pressure in the ventricular cavity and a pressure in the drainage chamber, and the second pressure differential is derived from a difference between a pressure in the drainage chamber and a pressure in an adjacent area of the subject's brain.

A siphon control membrane is disposed between the drainage chamber and the adjacent area of the subject's brain. The second force is transmitted through a compression member disposed between the siphon control membrane and the fluid flow control member. A bias means is configured to bias a linear offset of the compression member.

The fluid flow control member may comprise a pressure control membrane covering the port. In an alternative embodiment, the fluid flow control member comprises a sealing ball configured to seal the port when the aggregate of forces falls below a preset value.

### BRIEF DESCRIPTION OF THE DRAWINGS

The novel features of this invention will be best understood from the accompanying drawings, taken in conjunction with the accompanying description, in which similar characters refer to similar parts, and in which:

FIG. 1 is a cross-sectional view of the proportional siphon control device of the present invention, comprising a pressure control membrane.

FIG. 2 is a cross-sectional view of a second embodiment of the device of FIG. 1 utilizing a sealing ball valve.

FIG. 3 is a cross-sectional view of a third embodiment of the device of FIG. 1 utilizing a rigid compressive member.

### DESCRIPTION OF THE PREFERRED EMBODIMENTS

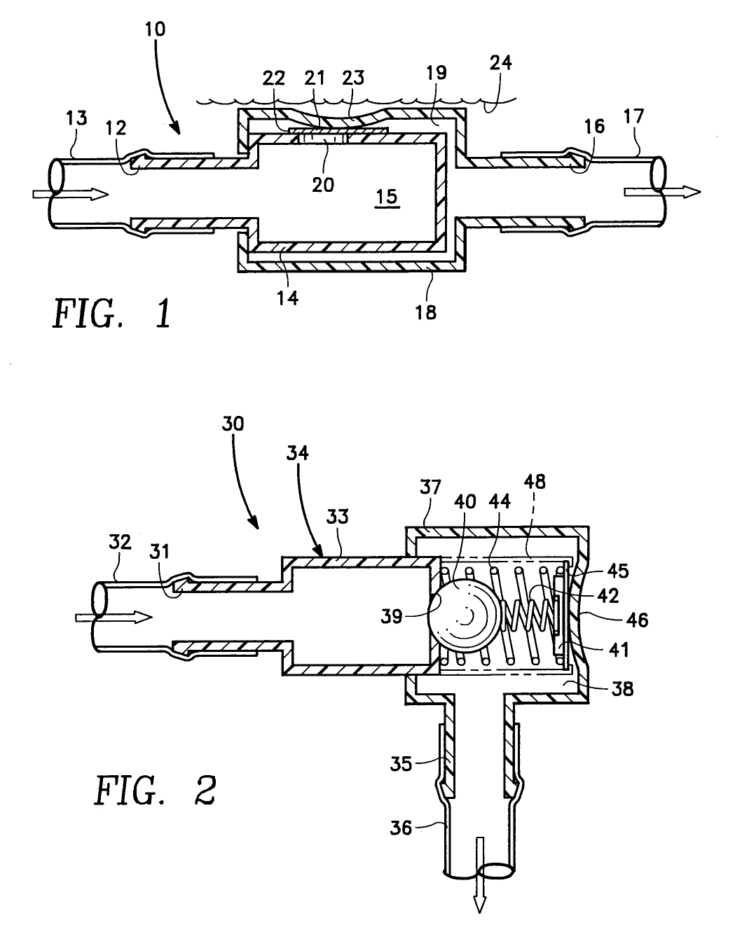

Referring now to a first embodiment of the present invention as depicted in FIG. 1, the proportional siphon control device of the present invention is shown and is generally designated by reference character 10. The device includes a ventricular nozzle 12 to which is attached a ventricular catheter 13. The ventricular catheter 13 has an outer end (not shown) insertable into a ventricle of the human brain. At the opposing inner end of FIG. 1, the ventricular catheter flares outwardly into a ventricular cavity wall 14 that defines a ventricular cavity 15. The ventricular cavity is in fluid communication with the ventricle nozzle 12.

A drainage catheter 17 is attached to a drainage nozzle 16. The drainage catheter 17 has an outer end (not shown) that is positioned in a body drainage region, preferably the torso peritoneal cavity. At an inner end, and as shown in FIG. 1, the drainage catheter 17 flares outward into a chamber wall 18 that is spaced-apart from, and corresponds in shape to, ventricular cavity wall 14. The annular space between the cavity wall 14 and chamber wall 18 define a drainage chamber 19. The drainage chamber 19 is in fluid communication with the drainage nozzle 16.

The ventricular cavity wall 14 is formed with at least one cavity port 20. In FIG. 1, the port has a round shape when viewed from above. It is to be appreciated, however, that other geometric shapes could be used for the port, and further that any number of ports could be placed in the cavity wall as required. The port 20 allows for fluid communication between the ventricular cavity 15 and the drainage chamber 19.

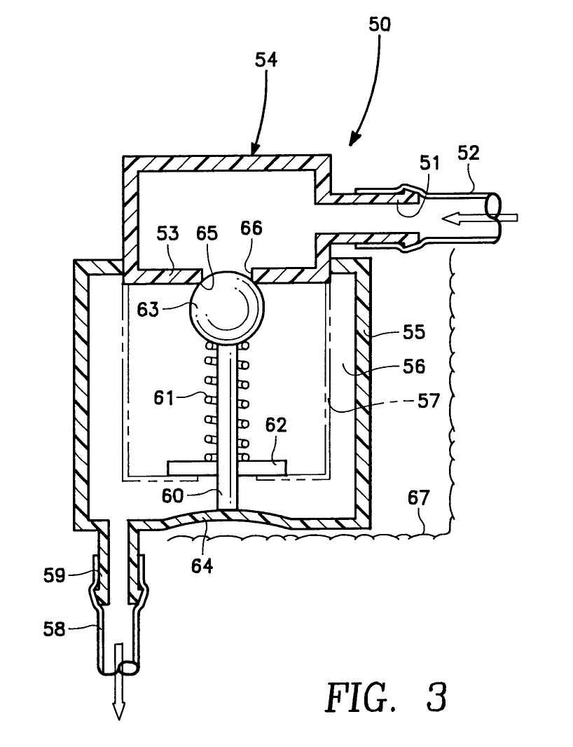

According to the embodiment of FIG. 1, the drainage chamber 19 substantially encloses the ventricular cavity 15 and the ventricular cavity wall 14. This can advantageously be accomplished if the drainage chamber substantially corresponds to the same shape as the ventricular cavity 15. It is to be appreciated, however, that the ventricular cavity does not have to be nested within the drainage chamber, as illustrated by the alternative embodiments depicted in FIGS. 2 and 3. As noted, the drainage chamber wall 18 merges into the drainage nozzle 16, and the drainage chamber is in fluid communication with the drainage nozzle 17 and the attached drainage catheter 16. The catheter, in turn, is in fluid communication with a patient's abdominal region or the heart.

The device of the present invention further includes at least one pressure control membrane, shown as a flexible flap 21. The number, size and shape of the pressure control membrane(s) corresponds to the number, size and shape of the respective port(s) 20. The pressure control flap 21 is positioned on the outer surface of the ventricular cavity wall 14 so that it covers the cavity wall port 20 and is fixed to the ventricular cavity wall 14 on at least one end 22 of the flap. In this manner, the pressure control flap may function as a swing-check valve.

As shown in FIG. 1, the drainage chamber wall 18 includes a flexible portion referred to as a siphon control membrane 23. The siphon control membrane 23 is positioned directly over flap 21, for reasons more fully described below.

During operation, once the CSF pressure in the brain reaches a predetermined point, the positive CSF pressure in the ventricular cavity 15 (which equals the CSF pressure in the brain) causes flap 21 to swing open away from port 20 and establish a path of fluid communication between the ventricular cavity 15 and the drainage chamber 19. As can be seen in FIG. 1, the complete flow path for CSF extends from the brain, through the ventricular catheter 13 and nozzle 12, into the ventricular cavity 15. Flow proceeds out port 20 through drainage chamber 19 and out the drainage nozzle 16 and catheter 17 into the peritoneal cavity, where the CSF is re-absorbed by the body. In this manner, the device of the present invention relieves CSF pressure on the brain.

Difference in height between the end of the drainage chamber 19 and the terminal end of the drainage catheter 17 in the peritoneal cavity, creates a negative pressure within the drainage chamber 19 relative to the surrounding brain area 24. This negative pressure can sometimes cause an unwanted siphoning effect of CSF from the brain. In the presence of negative pressure, however, the siphon control membrane 23 of the present invention flexes down onto the pressure control membrane shown as flap 21. In response, the flap 21 pivots about attached end 22 and is moved back against the outer surface of the ventricular cavity wall 14, until it contacts the wall and covers port 20.

If the two membranes tend to stick together, at least one of the membranes can be coated with a nonstick material or a layer of dissimilar material can be placed between them to prevent this. Alternatively, one or both of the membranes themselves can be made from dissimilar or nonstick materials. Once the port is covered, the flow path of CSF becomes blocked and the unwanted “siphoning” of CSF from the brain is eliminated or at least reduced.

The siphon control membrane 23 is a two-sided membrane. The interior side of the siphon control membrane is exposed to fluid pressure within the drainage chamber 19, and outer side of the siphon control membrane is exposed to an ambient pressure within the brain 24. As a result of the “siphon effect,” the pressure within the drainage chamber 19 can fall below the ambient fluid pressure of the brain on the opposite side of the siphon control membrane 23. As a result of this pressure differential, the siphon control membrane will flex inward.

The flexure of the siphon control membrane varies according to the amount of negative pressure present in the drainage catheter. Accordingly, the amount of resistive force transmitted by the siphon control membrane 46 to the pressure control membrane 23 varies in proportion to the inward flexure of the siphon control membrane 23. This resistive force thereby resists the opening of the pressure control membrane at the port 20.

As the pressure control membrane (flap) 21 is pressed more tightly against the periphery of the port, fluid flow through the port decreases. As the pressure control membrane 21 is released from the port 20, the opening of the port 20 increases, increasing fluid communication between the ventricular cavity 15 and the drainage chamber 19.

Accordingly, the shunt device varies the drainage effect according the amount of negative pressure that is present in the drainage catheter. The extent to which the siphon control membrane collapses onto the pressure control membrane, is proportional to the level of siphon suction experienced, thus creating a continuous balance.

Referring now to FIG. 2, a second embodiment of the proportional siphon control device of the present invention is shown and is generally designated by reference character 30. The device includes a ventricular nozzle 31 attached to a ventricular catheter 32. The ventricular catheter 32 has a terminal end (not shown) inserted into a ventricle of the human brain. The ventricular nozzle 31 merges into an outward flared ventricular enclosure wall 33 that defines a ventricular enclosure 34. The ventricular enclosure 34 is in fluid communication with the ventricle catheter 32.

A drainage nozzle 35 is attached to a drainage catheter 36. The drainage catheter 36 has a terminal end (not shown) extending into a drainage region of a patient, preferably the torso peritoneal cavity. The drainage nozzle 35 merges into an enlarged drainage compartment wall 37 that defines a drainage compartment 38. The drainage compartment 38 is in fluid communication with the drainage catheter 36.

Although the ventricular enclosure 34 of the embodiment of FIG. 2 is not enclosed within the drainage compartment 38, as in FIG. 1, this feature is largely stylistic. Therefore, those skilled in the art will readily understand that such structural features are largely interchangeable with respect to membrane, sealing ball, and other valve types.

With further reference to FIG. 2, a ventricular enclosure 34 is selectively placed in fluid communication with a drainage compartment 38 via selective occlusion of an enclosure port 39. To selectively occlude the port, however, this embodiment utilizes a sealing ball 40 and movable plate 41 that are interconnected by a pressure control spring 42. The pressure control spring is biased so that the sealing ball 40 is urged against the port 39 and blocks the port 39 in the absence of any positive overpressure of CSF in the ventricular cavity. To provide maximum sealing, the port 39 is round when viewed in plan. Additionally, the lip of the enclosure port may be beveled with a slight curvature configured to match the curvature of the sealing ball 40 when snugly pressed into the port 39.

A larger diameter compression mounting spring 44 is placed in a surrounding relationship with the sealing ball 40, the movable plate 41 and pressure control spring 42. One end of the mounting spring 44 rests against the ventricular enclosure wall 33, while the other opposing end of the mounting spring 44 rests against a mounting plate 45 that is positioned between the movable plate 41 and anti-siphon membrane 46 of drainage compartment 38. The mounting plate is spaced-apart from the underside of membrane 46 by an interior open structure 48 (depicted by phantom lines in FIG. 2). This structure will extend from enclosure walls 33 and engage the periphery of mounting plate 45. The mounting spring 44 has a spring constant that is greater than the spring constant of the inner pressure control spring 42.

During operation, when positive pressure in the ventricular enclosure 34 reaches a predetermined level, the pressure creates a force against the sealing ball 40 and compresses the pressure control spring 42. When this occurs, the sealing ball 40 is moved away from the enclosure port 39, and a path of fluid communication is established between the ventricular enclosure 34 and drainage compartment 38. This allows CSF to flow from the ventricular enclosure, through the enclosure port and into the drainage compartment. The CSF flows through the drainage compartment and into the drainage catheter 36 to the terminus of the drainage catheter 36, preferably proximate the peritoneal cavity as described above.

In the presence of negative pressure due to the aforementioned siphoning effect, the siphon control membrane 46 flexes inward toward the pressure control spring 42. This moves the mounting plate 45 away from engagement with interior structure 48 and toward the enclosure port 39, thereby further compressing both the mounting spring 44 and the inner pressure control spring 42. As the pressure control spring 42 is compressed, the spring counterforce is transmitted to sealing ball 40, thereby urging the sealing ball 40 against the enclosure port 39. When pressed snugly against the port 39, the sealing ball 40 will close the port opening, thereby preventing the above-referenced siphoning effect.

If the negative pressure is reduced, such as when a patient lies in a horizontal position, the anti-siphon membrane 46 returns to a “flat” unstressed state, thereby reducing the compressive force in the mounting spring 44 and pressure control spring 42. Because of an initial biasing on the springs 42, 44, a baseline compressive force on pressure control spring 42 will urge the sealing ball to remain against the port until positive pressure within the ventricular enclosure, again exceeds a predetermined set point.

The interior structure 48 maintains the pressure control spring in a proper orientation within drainage compartment 38. This arrangement permits the pressure control spring to regulate the distance between sealing ball 40 and the enclosure port 39, thereby regulating the flow of CSF through the port. The function of the mounting spring is also to constrain the ball into a position above the port and to maintain a biasing force on the mounting plate which is transmitted to the siphon control membrane 46.

FIG. 3 illustrates a third embodiment of the present invention, utilizing a rigid compressive member in place of the compression spring 42 of FIG. 2. CSF fluid is produced primarily within the lateral ventricle of the brain. A ventricular catheter 52 terminating in a subject's lateral ventricle (not shown), is attached to a ventricular inlet nozzle 51, through which CSF can flow into a ventricular enclosed receptacle 54 defined by receptacle walls 53. When receptacle port 66 is open, the CSF flows through the port into drainage space 56 defined by drainage housing walls 55, and out the drainage outlet 59 and drainage catheter 58. The drainage catheter may terminate in the peritoneal cavity of the patient, where the CSF can be safely discharged and reabsorbed by the body.

The sealing ball 63 can completely seal the receptacle port 66, or it can retract from the port, allowing CSF to flow through the port 66. Accordingly, the proximity of the sealing ball 63 to the port 66 determines the size of the opening in the port through which fluid can pass. Beveled edges 65 conforming to the curvature of the sealing ball 63 are advantageously formed on the ventricular receptacle wall 53 around the edge of the port 66, thereby forming a superior seal when the sealing ball 63 is pressed into the port 66.

A compression biasing spring 61 presses the sealing ball 63 into the receptacle port 66 with a constant pressure. The biasing spring coils can also act as a guide for an elongated rigid compressive member 60, so that the compressive member does not become dislodged. Similarly, the biasing spring can act as a retaining member for preventing the sealing ball 63 from becoming displaced from the port and rolling freely about within the drainage space 56.

As indicated above, the vertical posture of a subject is more likely to result in the process of siphoning, which will result in a negative pressure differential within the drainage space 56 relative to the pressure in the adjacent brain area 67. In response, housing siphon control membrane 64 will bow inwardly by an amount proportional to the negative pressure differential. This action will force the compressive member 60 against sealing ball 63 to close receptacle port 66. As with the first and second embodiments shown in FIGS. 1 and 2, a pressure differential between the ventricular receptacle 54 and the drainage space 56, urges the sealing ball 63 away from port 66, thereby increasing the cross sectional area through which CSF can flow through the port. Likewise, a pressure differential between the adjacent brain area 67 and the drainage space 56 urges the sealing ball 63 into the port 66, thereby reducing the cross sectional area through which CSF can flow.

As shown in FIG. 3, the biasing spring 61 rests against a collar 62, which is connected to a support structure 57. The support structure extends from a wall 53 of the ventricular receptacle 54. This arrangement of support elements creates a stable spring biasing action that is not subject to the same measure of change as a spring coupled to a siphon control membrane. The support structure 57 preferably has an open configuration so that it does not interfere with the flow of CSF or create a pressure differential between opposite sides of the support structure 57.

Although the respective combination of features in each of FIGS. 1-3 are in many cases preferred combinations, it is understood that many of the distinct features of FIGS. 1-3 are interchangeable. For example, structures represented by the terms “cavity,” “enclosure” and “receptacle” provide equivalent functions. Similarly, structures referenced as “chamber,” “compartment” and “space” provide equivalent functions. Still further, any of these structures can utilize different valve means such as flap membrane 21 of FIG. 1, or the sealing balls 40, 63 of FIGS. 2 and 3. Similarly, resisting forces can be transmitted to a transfer means such as flap membrane 21, mounting plate 45 and compressive member 60, directly from siphon control membranes 23, 46 and 64, through a dissimilar layer of material as discussed in conjunction with FIG. 1, a dissimilar layer of material as discussed in conjunction with FIG. 1, through a pressure control spring 42 as illustrated in FIG. 2, a rigid compressive member 60 as illustrated in FIG. 3, or any other structure or configuration capable of transmitting a force from the siphon control membrane 23 to a pressure control member or valve structure.

The approach described in FIGS. 1-3 provides a gradual or “analog” type of control, thereby avoiding the on/off cycling of prior art designs, maintaining a more uniform CSF pressure in the brain under a variety of different conditions. The present device can also be manufactured in a smaller overall size, which is desirable, since it must be implanted under the scalp.

The present invention also envisions an improved means for adjusting and biasing a hydrocephalus shunt valve. Because a certain ambient pressure in the CSF is desirable, it is important that the valve does not open at the first sign of positive pressure from the lateral ventricle to an ambient pressure in the brain, which is represented by the pressure differential between the ventricular cavity and the drainage cavity. Fluid communication between the ventricular cavity and the drainage cavity should only occur when the pressure differential between the ventricle cavity and the drainage cavity reaches a preset value. To achieve this control, a resistive biasing force must be applied to the pressure control device, such as the pressure control membrane 21 or the sealing balls 40, 63, preventing the valve from opening prematurely. Hydrocephalus shunt valves currently use a mechanical adjustment (moving member) to change the mechanical bias in a flexural member, which seals a port against a fluid pressure. The mechanical bias determines the pressure level at which the valve will open. The adjustment is generally made by applying a magnetic force that causes a ferromagnetic piece within the flexural member to move, which further alters the flexural bias of the valve.

The proposed innovation is to use an electromagnetic signal to adjust the mechanical bias by inducing resonant or other response in a circuit within the valve. This could be used to cause a material component to expand or cause a physical displacement of an electromagnetic component. The electromagnetic signal could be digital or analog radio waves, light applied through the scalp or any other form of electromagnetic radiation.

The advantages of the present invention include a method and apparatus for regulating a cerebrospinal fluid shunt that is responsive to the cerebrospinal fluid pressure in a patient while avoiding the siphoning effect that occurs when a drain is at a lower level than the shunt. The present invention is also directed to a cerebrospinal fluid shunt that operates in a graduated or analog fashion rather than an on/off fashion, and exhibits a greater cooperation between responses to cerebrospinal fluid pressure and resistance to siphoning.

It will be readily apparent to those skilled in the art that the present invention may be practiced without the use or incorporation of many of the specific details provided herein. Moreover, the present invention is intended to cover alternative designs, modifications, and equivalents which may be included within the spirit and scope of the foregoing invention. Accordingly, the various details added throughout the foregoing description should not be construed so as to limit the spirit and scope of the claims appended hereto.

I claim:

1. A pressure control valve for maintaining a proper cerebrospinal fluid pressure in the brain relative to ambient body pressure, comprising:
a) a ventricular cavity for receiving cerebrospinal fluid;
b) a drainage chamber for receiving cerebrospinal fluid from the ventricular cavity;
c) a port connecting the ventricular cavity to the drainage chamber such that the ventricular cavity and the drainage chamber are in fluid communion when the port is open;
d) a first pressure responsive member located adjacent said port for opening the port in an amount proportional to a first pressure differential, the first pressure differential being measured from a pressure in the ventricular cavity to a pressure in the drainage chamber; and,
e) a second pressure responsive member located adjacent said second pressure responsive member for acting upon said second pressure responsive member to resist an opening of the port in an amount proportional to a second pressure differential, the second pressure differential being measured from said ambient body pressure to a pressure in the drainage chamber.

2. The pressure control valve according to claim 1 wherein the first pressure responsive member is disposed within the drainage chamber.

3. The pressure control valve according to claim 1 wherein the first pressure responsive member includes a movable pressure control membrane covering the port.

4. The pressure control valve according to claim 3 wherein the second pressure responsive member comprises a siphon control membrane configured to impart a resistive force on the pressure control membrane, the resistive force being proportional to the second pressure differential.

5. The pressure control valve according to claim 4 further comprising a dissimilar material disposed between the siphon control membrane and the pressure control membrane.

6. The pressure control valve according to claim 1 wherein the first pressure responsive member includes a sealing ball configured to regulate a cross-sectional area through which the cerebrospinal fluid can flow through the port from the ventricular cavity to the drainage chamber according to an aggregation of forces acting on the sealing ball against the port, a first force being formed from the first pressure differential;
said pressure control valve further comprising a compressive member disposed between the sealing ball and the second pressure responsive member, the compressive member configured to impart a second force upon the sealing ball to press the sealing ball into the port to reduce fluid flow through said port.

7. The pressure control valve according to claim 6 whereby:
a) when the aggregation of forces is less than or equal to zero, the sealing ball will seat against the port such that no cerebrospinal fluid can pass through the port; and,
b) when the aggregation of forces exceeds a preset value, the sealing ball will be forced by fluid pressure to unseat from the port, thereby allowing a fluid flow through the port from the ventricular cavity to the drainage chamber.

8. The pressure control valve according to claim 6 wherein the compressive member is a spring.

9. The pressure control valve according to claim 6 wherein the compressive member is a substantially rigid member.

10. The pressure control valve according to claim 7 further comprising a base plate disposed between the second end of the compressive member and the second pressure responsive member.

11. The pressure control valve according to claim 10 further comprising a biasing member configured to control a displacement offset between the compressive member and the second pressure responsive member.

12. The pressure control valve according to claim 11 wherein the biasing member comprises a material selected from a group consisting of electrostrictive materials and magnetostrictive materials.

13. The pressure control valve according to claim 12 wherein the biasing member further includes a linear actuator.

14. The pressure control valve according to claim 12 wherein the biasing member further includes an oscillating device used to rotate a cam.

15. The pressure control valve according to claim 8 further including a second spring oriented such that the first spring is disposed within a cylindrical center of the second spring.

16. A method of controlling cerebrospinal fluid pressure in a brain of a subject using a pressure control assembly having a ventricular cavity and a drainage chamber which are in fluid communication with an orifice, wherein flow through said orifice is regulated by a fluid control member, comprising the steps of:
a) channeling cerebrospinal fluid from said brain to said ventricular cavity;
b) allowing a first fluid pressure to develop within said ventricular cavity;
c) exerting a first force on said fluid control member according to a first pressure differential between a fluid pressure in the ventricular cavity and a fluid pressure within the drainage chamber;
d) exerting a second force on the fluid control member according to a second pressure differential; and,
e) controlling a rate of fluid flow from the ventricular cavity to the drainage chamber through said orifice.

17. The method according to claim 16 wherein the rate of fluid flow is proportional to the first force, and inversely proportional to the second force.

18. The method according to claim 17 wherein the second pressure differential is related to the difference between an ambient pressure in the brain of the subject and the fluid pressure within the drainage chamber.

19. The method according to claim 16 wherein the fluid control member is a membrane that is movable relative to said orifice to regulate fluid flow through said orifice.

20. The method according to claim 16 wherein the fluid control member is a sealing ball configured to regulate fluid flow through the orifice.

21. The method according to claim 16 further comprising the step of biasing the second force through an adjustable offset bias means.

22. The method according to claim 21 wherein the adjustable offset bias means comprises a biasing material that is not subject to ferro-magnetic corrosion and deterioration.

23. The method according to claim 21 further comprising the step of generating a control signal to regulate the adjustable offset bias means.

24. The method according to claim 16 further comprising the step of draining the cerebrospinal fluid from the drainage chamber into an abdominal cavity of the subject.

25. A pressure control apparatus for maintaining a predetermined cerebrospinal fluid pressure within the brain of a subject, the apparatus comprising:
a) a ventricular cavity having an orifice;
b) a drainage chamber in fluid communication with said ventricular cavity through said orifice;
c) a catheter for channeling cerebrospinal fluid from the brain to a ventricular cavity; and,
d) a fluid flow control member in operative engagement with said orifice to regulate a size of an opening through the orifice according to an aggregate of a first and second force acting on the fluid flow control member, wherein a first force is related to a first pressure differential, and a second force is related to a second pressure differential.

26. The pressure control apparatus according to claim 25 wherein the first pressure differential is derived from a difference between a pressure in the ventricular cavity and a pressure in the drainage chamber.

27. The pressure control apparatus according to claim 25 wherein the second pressure differential is derived from a difference between a pressure in the drainage chamber and a pressure in an adjacent area of said brain.

28. The pressure control apparatus according to claim 27 comprising a siphon control membrane disposed between the drainage chamber and said adjacent area, wherein the second force is transmitted through a compression member disposed between the siphon control membrane and the fluid flow control member.

29. The apparatus according to claim 28 wherein the fluid flow control member includes a pressure control membrane covering said orifice.

30. The apparatus according to claim 28 wherein the fluid flow control member includes a sealing ball configured to close said orifice when the aggregate of forces falls below a preset value.

31. The apparatus according to claim 28 further comprising a bias means for biasing a linear offset of the compression member.

32. The pressure control valve according to claim 28 wherein the compression member includes a compression spring.

33. The pressure control valve according to claim 28 wherein the compression member includes a substantially rigid member.

34. The pressure control valve according to claim 28 further comprising a base plate disposed between the compression member and the siphon control membrane.
